(19) United States
(12) Patent Application Publication
(10) Pub. No.: US 2006/0160170 A1
(43) Pub. Date: Jul. 20, 2006

(54) Method and device of rapid antigen extraction

(76) Inventor: Paolo Giordano, Segrate (Milano) (IT)

Correspondence Address: DILWORTH & BARRESE, LLP, 333 EARLE OVINGTON BLVD., UNIONDALE, NY, 11553, US

(21) Appl. No.: 11/312,167

(22) Filed: Dec. 20, 2005

(30) Foreign Application Priority Data
Dec. 21, 2004 (IT) MI2004A002434

(51) Int. Cl. C12Q 1/04; C12M 1/34; G01N 33/554; G01N 33/569

(52) U.S. Cl. 435/34; 435/287.1; 435/7.32

(57) ABSTRACT

A system and a method of the interaction and preservation of two or more reagents used in a chemical reaction is described, in which the reagents are put in contact one with another only when a wad (A) carrying a sample to be treated with said reagents breaks a partition barrier (4, 14) placed between two containers belonging to a device comprising a first upper container (2, 12) and a second lower container (3, 13) of a test tube (1), the bottom wall of the first container (2, 12) forming said partition barrier (4, 14) able to be perforated by the wad (A) carrying the sample to be treated.

### BACKGROUND OF THE INVENTION

The present invention is about a method and a device for preserving two or more reagents used in a chemical reaction.

Different methods for preserving two or more reagents, in order to put them in contact only when they have to be used, are known in the state of the art. For example, in pharmaceutical preparations, a reagent is commonly preserved in a solid state and it is dissolved in a liquid state solvent just before the use, by perforating a mechanical partition wall.

Other preservation methods have been described as well, in which two or more containers, each containing a reagent, are inserted into a single main container inside which, after the breakage of the internal containers, the reagent mixing takes place.

The traditional methods of extraction of saccharidic antigens from group A streptococcus provide for the use of liquid reagents (Lenneft, E. H., Ed., Manual of Clinical Microbiology, Fourth Edition, American Society of Microbiology, Washington, D.C., 1985, pages 170-171). Typically, two liquid reagents are used in the extraction stage: an acid (acetic, hydrochloric or citric acid) and a sodium nitrite solution. The two reagents are mixed in a test tube in which the wad used to take the biological sample to be examined is inserted.

In other known methods, at least one of the two reagents is in a solid state (U.S. Pat. No. 5,536,646), or the reagents are contained in test tubes or flasks with several separate compartments, each containing a single reagent to be mixed immediately before use (U.S. Pat. No. 4,673,639).

By mixing the two reagents, nitrous acid, which is a relatively unstable acid, is produced, thus requiring that the reagents are mixed immediately before the extraction process starts. Otherwise, the instability of the resulting nitrous acid solution can reduce the extraction effectiveness. In fact, if the reagents are prematurely mixed with respect to the biological sample addition, according to what described in U.S. Pat. No. 4,851,337, the nitrous acid decomposition takes place and the extraction solution can lose its effectiveness in a very short time.

According to the method described in U.S. Pat. No. 5,415,994, the extraction takes place in a well directly obtained in the cartridge containing the immunochromatographic strip. One of the two reagents is contained in a flask, that contains in its turn a phial with the second reagent. The operator mixes the two reagents by breaking the phial and then pours the mixture in the well in which the wad is placed. In this way, the operator does not have to count the drop number of each reagent. However, in this case too the extraction takes place after the two reagents have been mixed. Moreover, the insertion of the wad in the well sometimes causes the even partial occlusion of the liquid drainage conduit, and thus the flow is slowed down or even blocked. Otherwise, if the wad is inserted in a manner such that the liquid does not flow therethrough before reaching the immunochromatographic chamber, the reaction liquid can reach the immunochromatographic membrane before the extraction takes place.

According to other known methods (U.S. Pat. No. 5,494,801), a third reagent is added to the two default ones in order to neutralize the solution before the chromatographic stage takes place. At present, however, the use of three reagents is considered too complicated for the operator, and thus the systems providing for the use of two reagents only are preferred. Furthermore, even these methods do not avoid the risk of effectiveness reduction of the extraction due to the time elapsed between the reagent mixing and the sample insertion.

Methods that provide for the insertion of the sample, on which the extraction has to be performed, in a device before adding the reagents have been described too (U.S. Pat. No. 6,168,956). In these methods, the operator must apply the reagents according to the optimal time sequence. However, the need to determine the volume of the reagents (for example, by counting the reagent drops) still remains, as well as the possibility to use the same reagent twice.

The traditional methods of extraction of lipopolysaccharidic antigens from Chlamydia trachomatis provide for the use of an alkaline reagent able to extract the lipopolysaccharidic antigens, in which an acid or a buffer is inserted to neutralize the extraction. According to a traditional method, the alkaline reagent consists of sodium hydroxide and the acid reagent for neutralizing the extraction solution is hydrochloric acid.

The wad through which the biological sample has been taken is inserted into a test tube containing the alkaline reagent for the extraction and it is agitated for a predetermined time, after which the neutralization reagent is added.

### SUMMARY OF THE INVENTION

It is thus an object of the present invention to overcome the problems of the aforementioned prior art method and devices.

The present invention can be applied in all the situations in which two or more reagents, that can start a chemical reaction together with a sample to be treated, can not be mixed together before adding the sample. For example, it is possible that the sample has to be put in contact with an unstable reaction product, or with more than one reagent, in a predetermined sequence.

More specifically, the present invention can be applied to the bacterial antigens extraction processes performed with wad on human or animal samples. In particular, the present invention can be used for the extraction of saccharidic antigens from group A and B streptococcus, as well as of lipopolysaccharidic antigens from Chlamydia.

The main object of the method and the device of rapid antigen extraction according to the present invention is thus to simplify the aforementioned prior art extraction processes, in particular:
a) the operator does not have to predetermine and check the reagent volume, for example by counting the dispensed reagent drops;
b) the time needed to dissolve one or more solid state reagents is not required;
c) the time elapsed between the reagent mixing and the insertion of the wad with the biological sample does not reduce the extraction effectiveness, for example when the highly unstable nitrous acid is used.

### BRIEF DESCRIPTION OF THE DRAWINGS

Further characteristics and advantages of the method of rapid antigen extraction according to the present invention, and the device for performing said method, will be better highlighted in the following description of preferred embodiments, given in an explanatory but not limiting way with reference to the annexed figures of drawings, in which:

FIG. 1 is a schematic sectional view of a first embodiment of a device for performing the method of rapid antigen extraction according to the present invention;

FIG. 2 is a schematic sectional view of a second embodiment of a device for performing the method of rapid antigen extraction according to the present invention; and

FIG. 3 is a schematic sectional view of an embodiment of a flexible extraction device inserted in a rigid tube for the breakage of the mechanical barrier between the two reagents.

### DESCRIPTION OF THE PREFERRED EMBODIMENTS

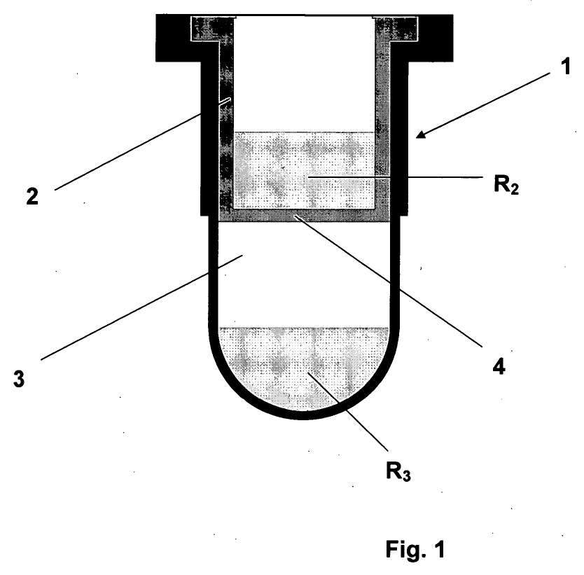

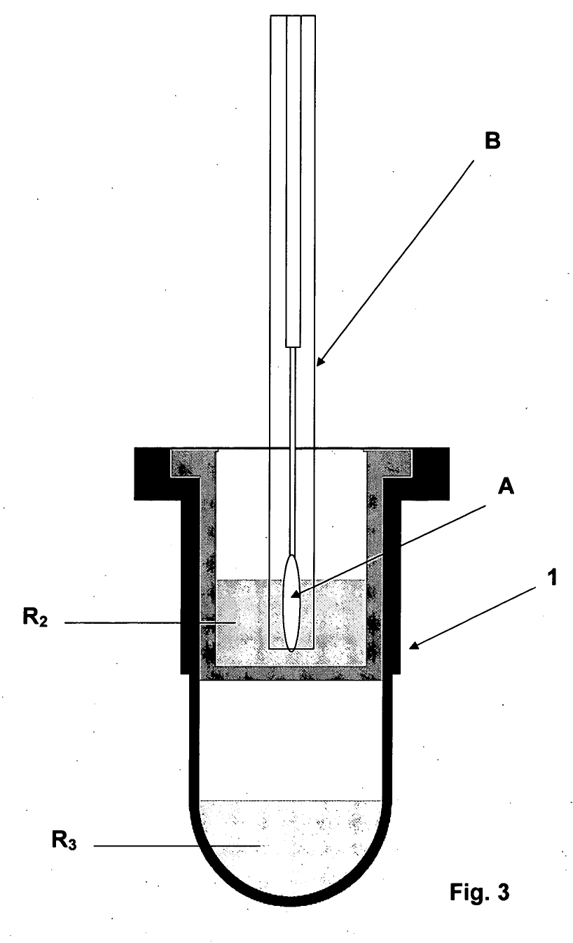

With reference to FIG. 1, according to the method of the invention, the two reagents are poured in predetermined volumes in a preferably but not necessarily cylindrical test tube 1 during the manufacturing stage of the device for performing said method, said test tube 1 comprising a first inner container 2 able to be inserted in a second outer container 3 that forms the main body of the test tube 1. The container 2 is configurated to form a cap for the container 3. The two containers 2 and 3 are then assembled so that the wad A (FIG. 3) with the sample to be tested sequentially passes through the reagent R₂, placed inside the container 2, and the reagent R₃, placed inside the container 3, respectively by breaking the mechanical barrier 4 that forms the bottom wall of said container 2 and separates said two reagents R₂ and R₃. The containers 2 and 3 can be manufactured with any kind of material compatible with the reagents R₂ and R₃ contained therein, and can have a proper shape and a sufficient volume to contain the wad A, said reagents R₂ and R₃ being in turn either in liquid or in solid state. The test tube 1 is sealed at its top using any known sealing system, for example with a metallic sheet (not shown).

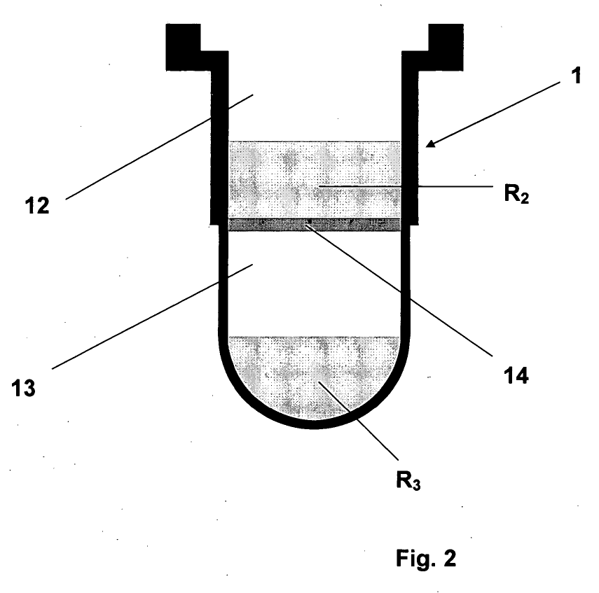

Another embodiment of the device for performing the method according to the present invention is shown in FIG. 2. After pouring the reagent R₃ in the test tube 1, it is possible to form a partition wall 14 in said test tube 1 having the same function of the container 2 mechanical barrier 4, for example by adding solid paraffin, heating it up to its melting and thus letting it cool down until it forms a proper physical partition element 14 similar to said mechanical barrier 4, thus being able to define two separate containers, an upper one 12 and a lower one 13, inside the same test tube 1. At this point it is possible to add the second reagent R₂ into the so formed upper container 12 of the test tube 1. In this case too the test tube 1 is subsequently sealed at its top using a known sealing system.

The device consisting of the test tube 1 for performing the method according to the invention is thus able to ensure that the two reagents R₂ and R₃ are put in contact only when the wad A bearing the sample is present. Therefore, according to the method of the invention, the transportation of the first reagent to the second reagent is performed by the wad A itself.

According to a preferred aspect of the invention, the sample is taken with a pharyngeal wad A following well known procedures. The wad A is then inserted into the first container 2 or 12 of the test tube 1, after the removal of the seal, and it is then driven in the second container 3 or 13, by breaking the barrier 4 between the containers 2 and 3 or the partition wall 14 between the containers 12 and 13. The extraction of the antigen by means of the so formed nitrous acid is thus started. When the expected extraction time is lapsed, the wad A is removed, preferably with the first container 2 if present, from the test tube 1 and the liquid can be poured directly from said test tube 1 into the immunochromatographic device for the antigen detection.

For example, according to the method of the invention, the reagent R₂ contained in the first container 2, 12 can be a 0.4 M acetic acid. The operator inserts the wad A into the first container 2, 12 of the test tube 1. The reagent R₂ is almost fully absorbed by the wad A. By pushing the wad A against the bottom 4, 14 of the container 2, 12, said bottom 4, 14 breaks, allowing said wad A to pass through and thus to reach the reagent R₃, for instance a 2 M sodium nitrite, in the second container 3, 13. At this point, the nitrous acid formation reaction takes place, due to the antigen presence on the wad A. Therefore, the antigen extraction takes place in the best conditions for the effectiveness of said extraction. Once the expected time for the extraction of the bacterial antigens from the wad A is lapsed, the wad A is removed from the test tube 1 and the liquid can be poured in the immunochromatographic strip cartridge well.

Another example of the method according to the present invention allows to extract Chlamydia antigens from cervical or urethral wads. The sample is taken with a cervical or urethral wad according to well known procedures. The wad is then inserted into the first container 2, 12 of the test tube 1, after the removal of said test tube seal, and it is left in contact with the reagent R₂ for the required extraction time. Once the extraction is finished, the wad is pushed into the second container 3 by breaking the barrier 4, or into the second container 13 by breaking the partition wall 14, and it is put in contact with the neutralization reagent R₃.

In this case, the reagents comprise an alkaline reagent (R₂) and an acidic neutralization reagent (R₃).

According to a traditional method, the cervical or urethral wad is inserted into a test tube containing 5 drops of 0.2 N sodium hydroxide, and it is left in the solution for 2 minutes. After shaking the wad, a predetermined volume of 0.1 N hydrochloric acid is added to neutralize the extraction solution. After shaking the wad again, said wad is then removed and a certain volume of the extraction solution is added to the test cartridge.

In order to take biological samples from particular sites, for example from the nasal cavities or the urethra, devices having a flexible and thin structure are available on the market, thus being difficult to break the partition wall between the two reagents with said devices. In this case, the sampling device can be inserted in advance into an assembly provided with the proper stiffness and resistance features for breaking the partition wall. For example, after the insertion of the sampling wad A into the test tube 1, it is possible to surround said wad A with a tube B having a proper diameter. By pushing the tube B, that breaks the barrier 4, the wad A is transported into the container 3, 13 (see FIG. 3).

It should be understood that several modifications could be made to the device, formed by the test tube 1, that performs the method of rapid antigen extraction extraction according to the present invention, as it is also defined in the appended claims. For example, the sealing of the test tube 1 can be obtained by using a cap, or by thermal sealing with an aluminium sheet coupled with polyethylene. Furthermore, although in the description the sample collection system is indicated as a “wad”, this denomination is merely used for convenience, since the most common systems for taking the A group streptococcus are the pharyngeal wads, while the most common systems for taking the Chlamydia trachomatis are the cervical or urethral wads. Therefore, it should be obvious for a man skilled in the art that it is possible to use any sampling system compatible with the immunological array format.

1. A method for preserving two or more reagents used in a chemical reaction, comprising the following steps:
inserting a wad (A), through which a chemical substance sample to be examined has been taken, into a first container (2, 12) belonging to a test tube (1) and containing a first reagent (R₂), said wad (A) carrying said chemical substance sample being able to at least partially absorb said first reagent (R₂) within a predetermined time period;
exerting a pressure with said wad (A), after the at least partially absorption of said first reagent (R₂) by said wad (A) carrying said chemical substance sample, against the bottom wall (4, 14) of said first container (2, 12), causing said wad (A) to break said bottom wall (4, 14) and to pass through said bottom wall (4, 14);
bringing said wad (A), carrying said chemical substance sample and at least a part of said first reagent (R₂), in contact with at least a second reagent (R₃) contained into a second container (3, 13) belonging to said test tube (1), ensuring said first reagent (R₂) and said second reagent (R₃) are put in contact one with another and said chemical substance sample only when said wad (A) carrying said chemical substance sample to be examined is present; and removing said wad (A) from said second container (3, 13) belonging to said test tube (1) after a predetermined time period and once the reaction between said first reagent (R₂), said second reagent (R₃) and said chemical substance sample is completed.

2. The method according to claim 1, wherein said first reagent (R₂) and said second reagent (R₃) are poured in predetermined volumes into said first container (2, 12) and into said second container (3, 13) respectively during the manufacturing stage of said test tube (1).

3. The method according to claim 1, wherein said wad (A) sequentially passes through said first reagent (R₂) and said second reagent (R₃).

4. The method according to claim 1, wherein said first reagent (R₂) and said second reagent (R₃) are able to form nitrous acid.

5. The method according to claim 1, wherein said second reagent (R₃) is in a solid state.

6. The method according to claim 1, wherein said wad (A) is transported through said bottom wall (4) by a rigid device (B) able to break said bottom wall (4).

7. The method according to claim 1, wherein said chemical substance sample to be examined remains in said first reagent (R₂) for a predetermined time period before being transferred into said second reagent (R₃).

8. The method according to claim 7, wherein said first reagent (R₂) and said second reagent (R₃) are a base and a neutralization solution respectively.

9. The method according to claim 7, wherein said first reagent (R₂) and said second reagent (R₃) are an acid and a neutralization solution respectively.

10. The method according to claim 1, wherein the removal of said wad (A) from said second container (3) belonging to said test tube (1) involves removal of said first container (2) from said test tube (1).

11. A device for performing the method of preserving two or more reagents used in a chemical reaction according to claim 1, comprising at least a first inner container (2) able to be inserted into at least a second outer container (3) belonging to a test tube (1), said at least a first container (2) having a bottom wall (4) able to be perforated by said wad (A) carrying said chemical substance sample.

12. A device for performing the method of preserving two or more reagents used in a chemical reaction according to claim 1, comprising a test tube (1) inside which at least a first upper container (12) and at least a second lower container (13) are defined, said at least a first container (12) having a bottom wall (14) made of a solid paraffin layer able to be perforated by said wad (A) carrying said chemical substance sample.

13. The device according to claim 11, wherein said second container (3, 13) forms the main body of said test tube (1).

14. The device according to claim 11, wherein said first container (2) is configurated to form a cap for said second container (3).

15. The device according to claim 11, wherein said test tube (1) is sealed by thermal sealing with an aluminium sheet coupled with polyethylene.

16. The device according to claim 11, wherein said test tube (1) is sealed by thermal sealing with a paper sheet coupled with polyethylene.

17. The device according to claim 11, wherein said first container (2) and said second container (3) are manufactured with any kind of material compatible with said first reagent (R₂) and said second reagent (R₃) contained therein, and can have a proper shape and a sufficient volume to contain said sampling wad (A), said first reagent (R₂) and said second reagent (R₃).

18. The device according to claim 12, wherein said test tube (1) is manufactured with any kind of material compatible with said first reagent (R₂) and said second reagent (R₃) contained therein, and can have a proper shape and a sufficient volume to contain said sampling wad (A), said first reagent (R₂) and said second reagent (R₃).

19. Use of the method according to claim 4 for the extraction of saccharidic antigens from group A and group B streptococcus, taken with a wad (A) or with other sampling systems.

20. Use of the method according to claim 8 for the extraction of lipopolysaccharidic antigens from Chlamydia trachomatis, taken with a wad (A) or with other sampling systems.

21. Use of the method with the device of claim 11 for the extraction of saccharidic antigens from group A and group B streptococcus and lipopolysaccharidic antigens from Chlamydia trachomatis, taken with a wad (A) or with other sampling systems.

22. The device according to claim 12, wherein said second container (3,13) forms the main body of said test tube (1).

23. The device according to claim 12, wherein said test tube (1) is sealed by thermal sealing with an aluminium sheet coupled with polyethylene.

24. The device according to claim 12, wherein said test tube (1) is sealed by thermal sealing with a paper sheet coupled with polyethylene.

25. Use of the method according to claim 9 for the extraction of lipopolysaccharidic antigens from Chlamydia trachomatis, taken with a wad (A) or with other sampling systems.

26. Use of the method with the device of claim 12 for the extraction of saccharidic antigens from group A and group B streptococcus and lipopolysaccharidic antigens from Chlamydia trachomatis, taken with a wad (A) or with other sampling systems.
